(19) United States
(12) Patent Application Publication
(10) Pub. No.: US 2011/0076684 A1
(43) Pub. Date: Mar. 31, 2011

(54) APPARATUS AND METHOD FOR ACQUIRING, DETECTING, AND ANALYZING CELLS IN A MICROFLUIDIC SYSTEM

(76) Inventors: Alexander FREY, Munchen (DE); Karsten Hiltawsky, Schwerte (DE); Daniel Sickert, Munchen (DE)

(21) Appl. No.: 12/880,379

(22) Filed: Sep. 13, 2010

(30) Foreign Application Priority Data
Sep. 29, 2009 (DE) 10 2009 043 426.7

(51) Int. Cl. C12Q 1/68; C12M 1/34; G01N 33/53; G01N 33/574; C12Q 1/02

(52) U.S. Cl. 435/6; 435/287.3; 435/7.21; 435/7.23; 435/29

(57) ABSTRACT

An apparatus is disclosed for acquiring, detecting, and, optionally, analyzing cells in a biological liquid, which apparatus has a microfluidic system. In at least one embodiment, the apparatus includes at least one microfluidic channel having a small cross-section which is completely or partially coated with cell-specific binding molecules and which has no flow obstacles dividing the fluid flow; at least one apparatus for generating a steady and alterable liquid flow through the microfluidic channel, and optionally at least one apparatus for analyzing the genetic information of the cell. In at least one embodiment, the method includes a sample of a biological liquid being conducted through an apparatus defined above at a defined velocity V₁ so that cells possibly present in the sample are bound to the cell-specific binding molecules; the sample of the biological liquid and/or a suitable liquid being conducted one or more times through the apparatus at a (different) defined velocity V₂ so that nonspecifically bound constituents of the sample are removed; the cells bound to the cell-specific binding molecules being appropriately detected; and optionally the cells bound in the channel being released and the genetic information of the cells being optionally analyzed, with the ratio of the velocities, V₁:V₂, being in the range from 1:5 to 1:10.

### PRIORITY STATEMENT

The present application hereby claims priority under 35 U.S.C. §119 on German patent application number DE 10 2009 043 426.7 filed Sep. 29, 2009, the entire contents of which are hereby incorporated herein by reference.

### FIELD

At least one embodiment of the present invention generally relates to an apparatus for acquiring, detecting, and analyzing cells in a biological liquid, which apparatus has a microfluidic system. At least one embodiment also generally relates a method for acquiring and, optionally, analyzing cells in a biological liquid using a microfluidic system. The method according to at least one embodiment of the invention is intended in particular for acquiring and analyzing circulating tumor cells and is thus preferably used in tumor diagnostics.

### BACKGROUND

A patient who suffers from a malignant cancer generally passes through various disease stages which depend on the type, size, and growth of the tumor. Characteristic of a malignant tumor is its unregulated and progressive, invasive growth which exceeds, from a certain stage, the original boundary of the organ. Typically, this organ-transcending growth leads, in the late phase of a cancer, to the formation of metastases, i.e., a formation of new tumor tissue at sites in the body other than the original primary tumor location.

Meanwhile, it is known that these metastases are caused by “circulating” tumor cells. The circulating tumor cells are released from the primary tumor and circulate in the blood through the body, whereby the formation of metastases can occur. The number of circulating tumor cells is used nowadays as a new parameter for prognosing the disease progression in patients who suffer from a metastasizing tumor. When the number of circulating tumor cells decreases with therapy, this is an indicator of successful therapy in terms of a regression of the metastases or the primary tumor. Thus, the detection of circulating tumor cells can also be used for therapy optimization.

Systematically finding these cells in early tumor stages is possible with methods known to date, but only with limited accuracy. A major challenge in testing blood samples consists in the low number of circulating tumor cells. A test method therefore has to be sensitive, such that one tumor cell is detected per milliliter of blood. At the same time, the method has to be very specific, since one milliliter of blood contains, inter alia, about ten million leukocytes which have partially similar properties to circulating tumor cells with regard to, for example, size, nucleus, etc., and which have, in part, similar surface properties as circulating tumor cells.

Since 2008, Veridex LLC has made commercially available for the detection of circulating tumor cells a product (CellSearch™) with which the number of circulating tumor cells in a blood sample can be determined and the tumor cells as such can be identified. The maximum volume of the blood sample to be tested with this system is 7.5 ml. The CellSearch™ system is based on the use of anti-EpCAM antibodies which are bonded to microscopically small, magnetic iron particulates. Use is made of the fact that most circulating tumor cells have the EpCAM antigen on their surface. With the help of the magnetic anti-EpCAM particulates, the tumor cells are magnetically concentrated in a first procedural step.

In a further procedural step, the concentrated circulating tumor cells are stained and examined by microscopy. Currently, the method from Veridex LLC has been approved only as an indicator of the disease progression in patients having a metastasizing tumor condition for breast cancer and prostate cancer. The method appears to be rather unsuitable for diagnosing early stages of a cancer (with distinctly fewer circulating tumor cells). First, the maximum blood volume that can be processed is limited to 7.5 ml, and has to be acquired in one blood withdrawal. Blood withdrawal is, per se, a methodical disadvantage when searching for a few circulating cells because only a small amount can be obtained from the 5,000 ml of blood typically available. As a result, a large inaccuracy in measurement arises owing to statistical variations (Poisson distribution) alone. Furthermore, other tests have meanwhile shown that a maximum yield cannot be achieved with the enrichment method which is used in the CellSearch™ test and which uses magnetic beads (cf. Nagrath, Nature 450(7173): 1235-1239, 2007).

WO/2008/130977 describes a method for sorting particles (such as, for example, cells) through an uncoated microchannel structure. Cells are sorted by exploiting fluidic properties of the particles. However, it is doubtful whether, in this way, a sufficient specificity with regard to distinguishing leukocytes from circulating tumor cells can be achieved.

Currently, biochips having a microchannel structure are in use, e.g., the chip from febit. This chip is, however, not perfused with liquids having solid constituents (such as cells or bacteria), but with liquids which comprise, in particular, nucleic acids and/or nucleotides. Furthermore, this chip is designed and chemically prepared such that it can be equipped with nucleotide chains. When equipping with nucleotide chains, it should be noted that a location-dependent variation of the nucleotide chains is highly desirable, because these serve as capture molecules during subsequent flushing of the channels. In this way, different nucleic acids can be captured and detected in a location-dependent manner. A homogeneous coating with a single nucleic acid is currently not used in practice.

Also, it has to be ensured with this type of nucleic acid detection that the flushed liquid does not comprise any cellular constituents. Therefore, it is not suggested to design the coating of the channels such that cellular constituents can specifically bind to the wall (e.g., via proteins/antibodies). Furthermore, there is also no attempt with this type of detection to emulate classical immunoassays, and therefore the coating of the chip channels with antibodies is likewise non-obvious. In particular, the introduction of antibodies, such as anti-EpCAM, is neither intended nor known.

None of the methods for finding circulating cells from the prior art currently delivers satisfactory results with regard to the cells to be found and, in particular, with regard to detection of circulating tumor cells in blood.

Furthermore, the detection of cells other than circulating tumor cells in blood also provides valuable diagnostic information. For example, the detection of circulating stem cells, circulating fetal cells, or circulating bacterial cells in a body fluid can provide diagnostic and prognostic information about the state of a patient.

### SUMMARY

The inventors discovered that there is thus a need for a method and an apparatus for acquiring, detecting, and analyzing cells in a biological liquid, in particular circulating tumor cells in a biological liquid, and this is thus provided by the invention. The method according to at least one embodiment of the invention shall be suitable for acquiring and analyzing any cells and, in particular, any circulating tumor cells in a biological liquid. It shall, in particular, be suitable for acquiring and detecting circulating tumor cells at an early stage of the disease, i.e., it shall allow the acquisition of circulating tumor cells at extremely low blood concentrations. The method and the apparatus, respectively, shall exhibit a high sensitivity and a high specificity.

Furthermore, the apparatus according to at least one embodiment of the invention and the method according to at least one embodiment of the invention, respectively, shall be easy to handle and suitable for automation. The apparatus shall further allow the binding of acquired and immobilized cells with specific protein markers so that the cells can be subsequently characterized using molecular biology techniques. It shall further be possible to recover the immobilized cells and to elute them with increased purity and concentration. It shall further be possible to acquire the nucleic acids of the immobilized cells. Overall, it shall be possible to characterize the cells in question.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

It was found that, surprisingly, whole cells can be specifically concentrated and detected with a microfluidic channel structure which has a small cross-section, which is completely or partially coated with cell-specific binding molecules, and which has no flow obstacles dividing the fluid flow. To this end, it is necessary for the cell suspension (e.g., blood) to have a slow fluid movement, during which the cells specifically bind to the wall of the coated channel structure.

An embodiment of the invention thus relates to an apparatus for acquiring, detecting, and, optionally, analyzing cells in a biological liquid, which apparatus has a microfluidic system and comprises a) at least one microfluidic channel having a small cross-section which is completely or partially coated with cell-specific binding molecules and which has no flow obstacles dividing the fluid flow, b) at least one apparatus, for generating a steady and alterable liquid flow through the microfluidic channel, and optionally c) at least one apparatus for analyzing the genetic information of the cells, and also to a method for acquiring, detecting, and, optionally, analyzing cells in a biological liquid, characterized in that a) a sample of a biological liquid is conducted through an apparatus defined above at a defined velocity V₁ so that cells possibly present in the sample are bound to the cell-specific binding molecules, b) the sample of the biological liquid and/or a suitable liquid is conducted one or more times through the apparatus at a defined velocity V₂ so that nonspecifically bound constituents of the sample are removed, c) the cells bound to the cell-specific binding molecules are appropriately detected, and optionally d) the bound cells are released and the genetic information of the cells is optionally analyzed, with the ratio of the velocities, V₁:V₂, being in the range from 1:5 to 1:10. The invention further relates to the abovementioned apparatus for use in diagnosing and/or prognosing the stage of a malignant cancer or for use in making decisions about the therapy or adapting the therapy in the case of a malignant cancer or during the disease progression. The invention further relates to a kit which comprises the abovementioned apparatus together with reagents for detecting the cells and/or for analyzing the genetic information of a cell.

An embodiment of the invention is based on the use of a microfluidic system. Microfluidic systems are central handling systems for fluids, such as liquids or gases, with or without a solids content in microtechnology or nanotechnology. They are used in particular in the area of biomedicine. In the art, numerous instruments with which microfluidic systems can be handled are known and commercially available. Mass transfer in fluidic systems is usually achieved through movement of the fluid, whereby the substances present therein are transported along. Particle movement depends not only on the geometric sizes and the acting surface forces but also in particular on the flow velocity of the fluid. Fluid movement usually takes place in channel systems, with the flow velocities usually being about 1-10 mm/s. The velocity of the particles in the fluid channels depends on the size of the channel cross-section, on the ratio of channel size to particle size, on the fluid velocity, on the adhesion of the particles to the channel walls, and on the shape of the particles.

An essential component of the apparatus according to an embodiment of the invention is thus a channel structure having a small cross-section or diameter (microchannel structure). Preferably, the cross-sectional area is smaller than 0.04 mm² for a round cross-section, smaller than 1 mm² for a rectangular cross-section, preferably smaller than 0.1 mm², more preferably in the range from 0.01 to 0.05 mm². The cross-section can be of any shape suitable for the purposes of the invention. Preferably, the cross-section is round, oval, or rectangular. It is advantageous, in the case of a rectangular-shaped channel, for the width-to-height ratio of the channel to be high. The width-to-height ratio of the channel should be in the range from 1:1 to 1000:1, preferably from 1:1 to 100:1, and particularly preferably from 5:1 to 20:1. In the case of a round cross-section, the diameter of this channel structure should be smaller than 200 μm; preferably, it is in the range from 50 to 100 μm. In the case of a rectangular cross-section, the height of a rectangular cross-sectional side should be smaller than 200 μm and preferably be in the range from 40 to 100 μm.

In an example embodiment, the width of the rectangular channel can be, for example; 220 μm, whereas the height is, for example, 65 μm. The cross-sectional width of the rectangular cross-section can be freely chosen from between 100 μm and 10 mm. The product of cross-sectional height and cross-sectional width determines the cross-sectional area, which determines the maximum achievable flow rate. The channel cross-section is designed such that there forms a flow profile which causes as many cells as possible to have contact at least once with the coated surface of the channel structure when passing through the channel structure. By introducing curves or constrictions, additional mixing processes can be achieved to result in every cell having contact at least once with the channel surface when completely passing through the channel structure. It is further essential that the channel structure has no flow obstacles dividing the fluid flow.

The surface of the channel structure is completely or partially coated with cell-specific binding molecules. These binding molecules are specifically directed against typical surface structures of the cells to be detected, and therefore these cells can be specifically bound to the wall of the channel structure. Cell-specific binding molecules are, for example, monoclonal or polyclonal antibodies, an antigen-binding fragment thereof, recombinant binding proteins, or an aptamer, or mixtures of binding molecules of this kind. Depending on the cell type to be detected, these binding molecules are directed against specific surface antigens or membrane proteins and transmembrane proteins. An example of this is the anti-EpCAM antibody which binds to circulating tumor cells. Further examples are antibodies to cytokeratins and typical epithelial proteins. Examples are CK5, CK6, CK8, CK17, CK18, Ber-EP4, Her-2/neu, MCSP, CEA, EMA, MUC1.

Techniques for producing appropriate binding molecules are well known in the art. Similarly, techniques for fixing and distributing these binding molecules on the surface of a channel structure are known. The microchannel structure makes it possible for a large surface area to be subjected to a flow of the fluid medium to be tested. When, additionally, the cross-section of the microchannel structure is kept small and also a favorable curve shape of the microchannel structure is chosen, these measures result in a flow profile of a kind where all constituents of the fluid medium have contact with the surface during passage through the microchannel structure. A small and flat cross-sectional area having a high width-to-height ratio is particularly advantageous, as explained above.

The microchannel structure can be produced from any materials which are compatible with biological liquids. Appropriate materials and also methods for producing such microchannels are known in the art.

The microchannel structure can have any shape. It can lead from entrance to exit in a straight path or in as many straight channel sections as desired, these being connected with one another via curved channel sections and it being possible to vary the curve as desired. Preference is given to the channel having a meander structure which runs semicircularly or circularly. The channel structure has to have at least one exit and one entrance so that the liquid to be tested can be pumped through.

The apparatus according to an embodiment of the invention thus comprises a further apparatus for generating a steady and alterable liquid flow, for example, a pump suitable for this purpose. Pumps of this kind are known in the art. Preferably, a connected pump of this kind can pump the liquid to be tested at variable rates through the channel structure, i.e., it has a variable output. The liquid can be pumped from left to right or vice versa or circularly. The flow rate or the flow velocity of the cells which flow past the coated surface must not exceed a certain value for a specific binding to occur in the first place. In the case of circular pumping, the liquid can also be pumped through an intermediate reservoir.

Thus, in a first step, a sample of the biological liquid is conducted through the apparatus at a defined velocity V₁. This velocity is a slow velocity, which makes it possible for the cells to form a specific bond with the binding molecules. Preferably, the velocity V₁ is in the range of 0.1-1 mm per second. In a next step, either once more the sample of the biological liquid, optionally mixed with a suitable, cell-compatible liquid, or a suitable liquid as such is conducted through the channel structure at a defined velocity V₂. The velocity V₂ is higher than the velocity V₁ and is preferably in the range of 1-10 mm per second.

It was found that, surprisingly, the detachment rate of nonspecifically bound cells at rapid pumping is higher than the detachment rate of specifically bound cells, i.e., even with faster pumping of the liquid, a specifically bound cell is no longer released, but nonspecifically bound cells are released. The slow pumping step at the velocity V₁ thus increases the sensitivity of the method; the fast pumping step at the velocity V₂ increases the specificity of the method by releasing nonspecifically bound constituents. The pumping step at the velocity V₂ can be repeated one or more times.

It was not obvious that cells which have already formed a specific bond at a coated surface can be rinsed by a liquid at a considerably higher rate without breaking the bond. This effect can be used in order, for example, to achieve a higher processing rate of a defined amount of liquid or, for example, to repeatedly remove (to wash off) nonspecifically bound molecules from the surface. Preferably, the microchannel structure comprises no additional surface structuring so that the resulting flow profile is similar in many parts and can be used in order, for example, to specifically wash out nonspecifically bound cells from the microchannel structure at a varying flow rate.

To release nonspecifically bound constituents of the biological liquid in the channel, the following rinsing liquids can be used: buffer solutions whose salt content, pH, and additives possibly present do not damage cells. Even the biological sample itself can be conducted through the channel again.

The cells bound to cell-specific binding molecules are detected by, for example, fluorescent labels which are directed against the cells to be detected (for example, anti-EpCAM fluorescent particles, cytokeratin labels). As a result, even at a low spatial resolution of an optical detection unit, the presence of specifically bound circulating tumor cells can be checked (Sandwich principle). Use can be further made of fluorescent labels which are specifically directed against antigens on nonspecifically bound cells, mainly leukocytes, in order to increase the specificity of the method (these antigens are thus not found on the tumor cells, e.g., anti-CD45).

In the above configuration of the method, the specific binding of circulating tumor cells can be observed continuously and with the help of a microscope or a CCD camera as an optical detection unit. To this end, the channel structure has to be translucent and the cells have to be preferably stained or fluorescently labeled.

A translucent channel structure is, for example, achieved by a channel structure made of glass or a translucent polymer. Materials of this kind are known in the art.

In order to stain the cells in question, two methods can be used. The cells can be dyed beforehand when present in the biological liquid. However, a disadvantage here is that large amounts of staining medium are required. Therefore, the immobilized cells in the channel are preferably stained by flushing with a suitable staining medium, after conducting the sample through the channel and rinsing, and subsequently rinsing out excess staining medium with a wash solution. Staining media for biological cells are well known in the art, and the staining is carried out according to the instructions of the particular manufacturer.

According to an embodiment of the invention, any biological liquids can be tested for the presence of cells. Examples of biological liquids are whole blood, plasma, saliva, bronchial lavage, ascitic fluid, cerebrospinal fluid, enriched fractions from the above biological liquids, such as, for example, by density gradient centrifugation, hemolysis, or filtering acquired, enriched whole blood fractions, etc. Examples of the cells occurring in the biological liquid are circulating tumor cells, bacterial cells, fungal cells, embryonic cells, immune cells, stem cells, fetal cells, etc., but also cellular fragments thereof. Preferably, the cells are circulating tumor cells or bacterial cells.

Preparing the biological samples for conductance through the microchannel structure is well known to a person skilled in the art. It is essential that the viscosity of the sample is lowered in an appropriate manner such that the sample is able to flow through channels. For example, blood should be made uncoagulable.

In the case of a positive detection of cells, in particular circulating tumor cells, these can be disrupted and the genetic information of the cell (for example, DNA, mRNA, or miRNA) can be analyzed in the same microchannel structure or in a downstream microchannel structure. Appropriate detection apparatuses are known in the art.

Such a detection method can be—together with the microchannel structure and the pump apparatus—easily implemented on one chip, providing advantages in terms of processing and costs. In terms of processing, at least one processing step is saved, since the concentrated circulating cells or tumor cells do not have to be extracted from the microchannel structure for further analysis. With regard to costs, it is advantageous that a (relatively) expensive further analysis of the cells, more particularly of the circulating tumor cells (e.g., miRNA analysis), would be carried out on the same chip only if a positive detection of circulating tumor cells has occured at all. This is important because, especially in the screening process, probably only a few cases of a positive detection of circulating tumor cells can be expected, and therefore the first step in a detection method (detection of circulating tumor cells) has to be cost-effective. The second step in a detection method (e.g., characterizing the circulating tumor cells by means of miRNA analysis) makes sense particularly in the case of a positive detection of CTC and would then also justify higher costs.

The method according to an embodiment of the invention is preferably used for diagnosing and/or prognosing the stage of a malignant cancer or for making the first diagnosis. The method according to an embodiment of the invention is further preferably used, using the apparatus, for making decisions about the therapy or adapting the therapy in the case of a malignant cancer or during the disease progression. The method according to an embodiment of the invention is applicable to all metastasizing tumor types, in particular those of epithelial origin, i.e., applicable to all carcinomas and adenocarcinomas when, for example, an anti-EpCAM antibody is used for the coating of the channel structure. However, it is also suitable for detecting other tumor types of mesenchymal origin, such as, for example, sarcomas or leukemias, when the channel structure is coated with antibodies which are specifically directed against antigens of these tumor types.

The method according to an embodiment of the invention can also be further used to acquire and analyze fetal cells for detecting fetal anomalies. Similarly, it is suitable for detecting bacterial cells circulating in blood, for example, as an indication of a sepsis which is starting or already present.

Furthermore, the method according to an embodiment of the invention is suitable for acquiring autologous stem cells from blood for therapeutic purposes. With the method according to an embodiment of the invention, stem cells can be concentrated and extracted and be used therapeutically, for example, in scarred heart muscle tissue after myocardial infarction or in Parkinson's disease.

Furthermore, the method according to an embodiment of the invention can also be used to detect proteins which are expressed by a cell via fluorescently labeled antibody cells. This allows, for example, the tissue origin of a tumor to be located, making the method particularly suitable for early diagnosis of a cancer.

Generally, the method according to an embodiment of the invention is suitable for diagnosing any type of disease which is caused by cellular components or involves a change in cellular components in a body liquid.

The method can be performed on out-patients by a physician, but can also be carried out in a hospital. Thus, an embodiment of the invention also relates to a method for diagnosing, prognosing, and monitoring the therapy of the abovementioned diseases.

An embodiment of the invention also comprises a kit which comprises the apparatus according to at least one embodiment of the invention together with reagents for detecting the cells and/or for analyzing the genetic information of a cell (molecular diagnostics). Reagents of this kind and also their use are well known in the art and are used according to the instructions of the manufacturer.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combineable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An apparatus for acquiring and detecting cells in a biological liquid, the apparatus including a microfluidic system and comprising:
at least one microfluidic channel having a small cross-section, which is completely or partially coated with cell-specific binding molecules and which has no flow obstacles dividing fluid flow; and
at least one apparatus for generating a steady and alterable liquid flow through the microfluidic channel, and optionally.

2. The apparatus as claimed in claim 1, wherein a width-to-height ratio of the at least one channel is high.

3. The apparatus as claimed in claim 1, wherein the width-to-height ratio of the channel is in the range from 1000:1 to 1:1.

4. The apparatus as claimed in claim 1, wherein the cell-specific binding molecules are directed against a surface antigen of the cells and are selected from the group consisting of an antibody, an antigen-binding fragment thereof, a recombinant binding protein, or an aptamer, or mixtures thereof.

5. The apparatus as claimed in claim 1, wherein the cell-specific binding molecules are directed against circulating tumor cells.

6. The apparatus as claimed in claim 1, wherein the apparatus is formed as a microfluidic chip.

7. A method for acquiring and detecting cells in a biological liquid, the method comprising:
conducting a sample of a biological liquid through an apparatus at a defined velocity V₁ so that cells possibly present in the sample are bound to the cell-specific binding molecules;
conducting at least one of the sample of the biological liquid and a suitable liquid one or more times through the apparatus at a defined velocity V₂ so that nonspecifically bound constituents of the sample are removed; and
appropriately detecting the cells bound to the cell-specific binding molecules are appropriately detected.

8. The method as claimed in claim 7, wherein the sample of a biological liquid is selected from the group consisting of whole blood, plasma, saliva, bronchial lavage, ascitic fluid, cerebrospinal fluid, or enriched fractions thereof.

9. The method as claimed in claim 7, wherein the cell-specific binding molecule is directed against a surface antigen of the cells and is selected from the group consisting of an antibody, an antigen-binding fragment thereof, a recombinant binding protein, or an aptamer, or mixtures thereof.

10. The method as claimed in claim 7, wherein the bound cells are detected by means of staining or by using fluorescently labeled antibodies.

11. The method as claimed in claim 7, wherein the velocity V₁ is in the range from 0.1 to 1 mm/s.

12. The method as claimed in claim 7, wherein the velocity V₂ is in the range from 1 to 10 mm/s.

13. The method as claimed in claim 7, wherein the cell-specific binding molecule is directed against circulating tumor cells.

14. A method, comprising:
using an apparatus as claimed in claim 1, for acquiring and analyzing cells in a biological liquid.

15. The method as claimed in claim 14, wherein the cells are circulating tumor cells.

16. The method as claimed in claim 14, wherein the biological liquid is selected from the group consisting of whole blood, plasma, saliva, bronchial lavage, ascitic fluid, cerebrospinal fluid, or enriched fractions thereof.

17. A method, comprising:
using an apparatus as claimed in claim 1, for at least one of diagnosing and prognosing the stage of a malignant cancer and also for making the first diagnosis.

18. A method, comprising:
using an apparatus as claimed in claim 1, for making decisions about the therapy or adapting the therapy in the case of a malignant cancer or during the disease progression.

19. A kit, comprising:
an apparatus as claimed in claim 1; and
reagents for at least one of detecting the cells and for analyzing the genetic information of a cell.

20. The apparatus as claimed in claim 1, further comprising:
at least one apparatus for analyzing genetic information of the cells.

21. The apparatus as claimed in claim 2, wherein the width-to-height ratio of the channel is in the range from 1000:1 to 1:1.

22. The method as claimed in claim 7, further comprising:
releasing the bound cells and analyzing the genetic information of the cells, with the ratio of the velocities, V₁:V₂, being in the range from 1:5 to 1:10.

23. The method as claimed in claim 15, wherein the biological liquid is selected from the group consisting of whole blood, plasma, saliva, bronchial lavage, ascitic fluid, cerebrospinal fluid, or enriched fractions thereof.

24. A method for acquiring and detecting cells in a biological liquid, the method comprising:
conducting a sample of a biological liquid through an apparatus as claimed in claim 1, at a defined velocity V₁ so that cells possibly present in the sample are bound to the cell-specific binding molecules;
conducting at least one of the sample of the biological liquid and a suitable liquid one or more times through the apparatus at a defined velocity V₂ so that nonspecifically bound constituents of the sample are removed; and
appropriately detecting the cells bound to the cell-specific binding molecules are appropriately detected.
